(19) United States
(12) Patent Application Publication
(10) Pub. No.: US 2026/0207094 A1
(43) Pub. Date: Jul. 23, 2026

(54) CHEMICAL SENSORS

(71) Applicant: Cardiac Pacemakers, Inc., St Paul, MN (US)

(72) Inventors: Yingbo Li, Woodbury, MN (US); Michael J. Kane, St Paul, MN (US); Rick Dorn, Plymouth, MN (US); Joseph Thomas Delaney, JR., Minneapolis, MN (US); Nicolas Ball-Jones, Minneapolis, MN (US); Cristian Parisi, Amherst, MA (US)

(21) Appl. No.: 19/452,502

(22) Filed: Jan. 19, 2026

Related U.S. Application Data
(60) Provisional application No. 63/748,308, filed on Jan. 22, 2025.

(51) Int. Cl. A61B 5/1473; A61B 5/00

(52) U.S. Cl. CPC A61B 5/14735; A61B 5/0017; A61B 5/6861

(57) ABSTRACT

A chemical indicator includes a plurality of indicator beads having embedded therein ion-selective sensor molecules; and a hydrogel surrounding the plurality of indicator beads, the hydrogel including a hydrophilic polymer bonded to a crosslinked network.

### CROSS REFERENCE TO RELATED APPLICATION

The application claims priority to Provisional Application No. 63/748,308, filed Jan. 22, 2025, which is herein incorporated by reference in its entirety.

### TECHNICAL FIELD

The present disclosure relates to chemical sensors. More specifically, the present disclosure relates to hydrogel formation and/or barrier films for use in various chemical sensors.

### BACKGROUND

Chemical sensors can be used to measure patients' physiological parameters.

### SUMMARY

In Example 1, a chemical indicator includes a plurality of indicator beads having embedded therein ion-selective sensor molecules; and a hydrogel surrounding the plurality of indicator beads, the hydrogel including a hydrophilic polymer bonded to a crosslinked network.

In Example 2, the chemical indicator of Example 1, wherein the hydrophilic polymer comprises polyethylene glycol (PEG); wherein the hydrogel is formed by crosslinking a functionalized PEG with a crosslinker to form the crosslinked network.

In Example 3, the chemical indicator of Example 2, wherein the functionalized PEG is selected from the group consisting of a mono-functional PEG, a bi-functional PEG, a multi-functional PEG, and a functionalized star PEG.

In Example 4, the chemical indicator of either Example 2 or 3, wherein the crosslinker comprises an acrylamide selected from the group consisting of a mono-functional acrylamide monomer and a bi-functional acrylamide monomer.

In Example 5, the chemical indicator of Example 4, wherein the mono-functional acrylamide monomer is selected from the group consisting of N-Isopropylacrylamide, N,N-dimethylacrylamide, N-(3-(Dimethylamino)propyl)acrylamide, 6-Acrylamidohexanoic acid, N-(Tris(hydroxymethyl)methyl)acrylamide, N-(2-Aminoethyl)acrylamide hydrochloride, N-(2-Amino-2-oxoethyl)-2-propenamide, and Acryloylglycine.

In Example 6, the chemical indicator of Example 4, wherein the bi-functional acrylamide monomer is selected from the group consisting of 1,4-Bis(acryloyl(piperazine, Ethylenebisacrylamide, N,N′-(1,2-Dihydroxyethylene)bisacrylamide, N,N′-1,3-Propanediylbis(2-propenamide), N,N′-1,6-Hexanediylbis(2-propenamide), N,N′-1,4-Butanediylbis(2-propenamide), N,N′-(Oxybis(2,1-ethanediyloxy-3,1-propanediyl))bis(2-propenamide), and N,N′-(1,3-Phenylenebis(methylene))bis(2-propenamide).

In Example 7, the chemical indicator of Example 1, wherein the hydrogel is formed by crosslinking a first functionalized PEG with a second functionalized PEG to form the crosslinked network.

In Example 8, the chemical indicator of Example 7, wherein the first functionalized PEG is a mono-functional PEG acrylamide and the second functionalized PEG is a bi-functional PEG acrylamide.

In Example 9, the chemical indicator of either Example 2 or 3, wherein the crosslinker is a multi-arm activated crosslinker.

In Example 10, the chemical indicator of Example 9, wherein the multi-arm activated crosslinker comprises an activated ester or an amine.

In Example 11, the chemical indicator of either Example 2 or 3, wherein the crosslinker comprises a diisocyanate.

In Example 12, the chemical indicator of either Example 2 or 3, wherein the crosslinked network comprises a polyacrylate network, a polyacrylamide network, a polyamide network, or a polyurethane network.

In Example 13, a chemical indicator includes a plurality of indicator beads having embedded therein ion-selective sensor molecules; and a hydrogel surrounding the plurality of indicator beads, the hydrogel including a hydrophilic polymer interpenetrated with a crosslinked network; wherein the hydrogel is formed by polymerizing a functionalized PEG into the crosslinked network.

In Example 14, the chemical indicator of any one of Examples 14-17, wherein the crosslinked network comprises polyacrylates, polyacrylamide, polyamide, polyurethane, polyurea, or a combination thereof.

In Example 15, an apparatus includes an optical feedthrough that includes a bottom portion, a side wall portion surrounding a periphery of the bottom portion to create a well, and a first window and a second window formed through the bottom portion of the optical feedthrough; and a chemical sensor cassette that is at least partially positioned in the well and that includes a chemical sensor cassette including an optical reflector; and the chemical indicator of any one of Examples 1 to 14.

In Example 16, a chemical indicator includes a plurality of indicator beads having embedded therein ion-selective sensor molecules; and a hydrogel surrounding the plurality of indicator beads, the hydrogel including a hydrophilic polymer bonded to a crosslinked network.

In Example 17, the chemical indicator of Example 16, wherein the hydrophilic polymer comprises polyethylene glycol (PEG); wherein the hydrogel is formed by crosslinking a functionalized PEG with a crosslinker to form the crosslinked network.

In Example 18, the chemical indicator of Example 17, wherein the functionalized PEG is selected from the group consisting of a mono-functional PEG, a bi-functional PEG, a multi-functional PEG, and a functionalized star PEG.

In Example 19, the chemical indicator of Example 17, wherein the crosslinker comprises an acrylamide selected from the group consisting of a mono-functional acrylamide monomer and a bi-functional acrylamide monomer.

In Example 20, the chemical indicator of Example 19, wherein the mono-functional acrylamide monomer is selected from the group consisting of N-Isopropylacrylamide, N,N-dimethylacrylamide, N-(3-(Dimethylamino)propyl)acrylamide, 6-Acrylamidohexanoic acid, N-(Tris(hydroxymethyl)methyl)acrylamide, N-(2-Aminoethyl)acrylamide hydrochloride, N-(2-Amino-2-oxoethyl)-2-propenamide, and Acryloylglycine.

In Example 21, the chemical indicator of Example 19, wherein the bi-functional acrylamide monomer is selected from the group consisting of 1,4-Bis(acryloyl(piperazine, Ethylenebisacrylamide, N,N′-(1,2-Dihydroxyethylene)bisacrylamide, N,N′-1,3-Propanediylbis(2-propenamide), N,N′-1,6-Hexanediylbis(2-propenamide), N,N′-1,4-Butanediylbis(2-propenamide), N,N′-(Oxybis(2,1-ethanediyloxy-3,1-propanediyl))bis(2-propenamide), and N,N′-(1,3-Phenylenebis(methylene))bis(2-propenamide).

In Example 22, the chemical indicator of Example 16, wherein the hydrogel is formed by crosslinking a first functionalized PEG with a second functionalized PEG to form the crosslinked network.

In Example 23, the chemical indicator of Example 22, wherein the first functionalized PEG is a mono-functional PEG acrylamide and the second functionalized PEG is a bi-functional PEG acrylamide.

In Example 24, the chemical indicator of Example 17, wherein the crosslinker is a multi-arm activated crosslinker.

In Example 25, the chemical indicator of Example 24, wherein the multi-arm activated crosslinker comprises an activated ester.

In Example 26, the chemical indicator of Example 24, wherein the multi-arm activated crosslinker comprises an amine.

In Example 27, the chemical indicator of Example 17, wherein the crosslinker comprises a diisocyanate.

In Example 28, the chemical indicator of Example 17, wherein the crosslinked network comprises a polyacrylate network, a polyacrylamide network, a polyamide network, or a polyurethane network.

In Example 29, a chemical indicator includes a plurality of indicator beads having embedded therein ion-selective sensor molecules; and a hydrogel surrounding the plurality of indicator beads, the hydrogel including a hydrophilic polymer interpenetrated with a crosslinked network.

In Example 30, the chemical indicator of Example 29, wherein the hydrogel is formed by polymerizing a functionalized PEG into the crosslinked network.

In Example 31, the chemical indicator of Example 30, wherein the functionalized PEG is a functionalized star PEG having more than two arms.

In Example 32, the chemical indicator of Example 31, wherein the functionalized PEG is physically entangled in the crosslinked network through a hydrogen bond and/or a non-covalent bond.

In Example 33, the chemical indicator of Example 32, wherein the crosslinked network comprises polyacrylates, polyacrylamide, polyamide, polyurethane, polyurea, or a combination thereof.

In Example 34, a chemical sensor cassette includes an optical reflector; and the chemical indicator of Example 16.

In Example 35, an apparatus includes an optical feedthrough that includes a bottom portion, a side wall portion surrounding a periphery of the bottom portion to create a well, and a first window and a second window formed through the bottom portion of the optical feedthrough; and a chemical sensor cassette that is at least partially positioned in the well and that includes the chemical sensor cassette of Example 34.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration of a chemical sensing system, in accordance with certain instances of the present disclosure.

FIG. 2 shows an implantable medical device with a chemical sensor, in accordance with certain instances of the present disclosure.

FIG. 3 shows an exploded view of the implantable medical device of FIG. 2, in accordance with certain instances of the present disclosure.

FIG. 4A shows a schematic cross-sectional view of a chemical sensor cassette, in accordance with certain instances of the present disclosure.

FIG. 4B shows a schematic bottom view of the chemical sensor cassette of FIG. 4A, in accordance with certain instances of the present disclosure.

FIG. 4C shows a schematic top view of the chemical sensor cassette of FIG. 4A positioned in an optical feedthrough, in accordance with certain instances of the present disclosure.

FIG. 5 shows a schematic cross-sectional view of a chemical sensor cassette, in accordance with certain instances of the present disclosure.

FIGS. 6A-6B show a schematic crosslinked network of a hydrogel and a reaction of forming the network, in accordance with certain instances of the present disclosure.

FIGS. 7A-7B show a schematic crosslinked network of a hydrogel and a reaction of forming the network, in accordance with certain instances of the present disclosure.

FIGS. 8A-8B show a schematic crosslinked network of a hydrogel and a reaction of forming the network, in accordance with certain instances of the present disclosure.

FIGS. 9A-9D show a schematic crosslinked network of a hydrogel and a reaction of forming the network, in accordance with certain instances of the present disclosure.

FIG. 10 shows a schematic cross-sectional view of a chemical sensor cassette, in accordance with certain instances of the present disclosure.

FIG. 11 shows a schematic cross-sectional view of parts of a film-based sensor, in accordance with certain instances of the present disclosure.

FIG. 12 is a graph showing leaching of ion-selective sensor molecules from an indicator layer of a film-based sensor without a barrier film, in accordance with certain instances of the present disclosure.

FIG. 13 is a graph showing leaching, or lack thereof, of ion-selective sensor molecules from an indicator layer comparing different film-based sensors with or without a barrier film, in accordance with certain instances of the present disclosure.

FIG. 14 is a graph showing sensor response time comparing various film-based sensors with or without a barrier film, in accordance with certain instances of the present disclosure.

FIG. 15 is a flow diagram of an example method for making part of the chemical sensor, in accordance with certain instances of the present disclosure.

FIG. 16 shows an exemplary setup for using rod coating to form a film-based sensor, in accordance with certain instances of the present disclosure.

While the disclosed subject matter is amenable to various modifications and alternative forms, specific instances have been shown by way of example in the drawings and are described in detail below. The intention, however, is not to limit the disclosed subject matter to the particular instances described. On the contrary, the disclosed subject matter is intended to cover all modifications, equivalents, and alternatives falling within the scope of the disclosed subject matter as defined by the appended claims.

### DETAILED DESCRIPTION

Physiological parameters such as concentrations of certain analytes (e.g., levels of potassium, sodium, creatinine, and other analytes) can be measured and monitored to evaluate various physical conditions and performance such as a person's kidney and/or cardiac conditions and performance.

Typically, measuring a person's analyte concentrations requires drawing multiple blood samples from a patient at a clinic and then processing the blood samples at a laboratory. One approach for measuring analyte concentrations that does not require periodic blood draws, etc., is to use an implantable chemical sensor. An implantable chemical sensor can use opto-electronic components such as light emitters and light detectors to sense one or more optical properties of chemical-based sensors. Optical properties of the chemical-based sensors can used to estimate analyte concentrations.

### Chemical Sensing System

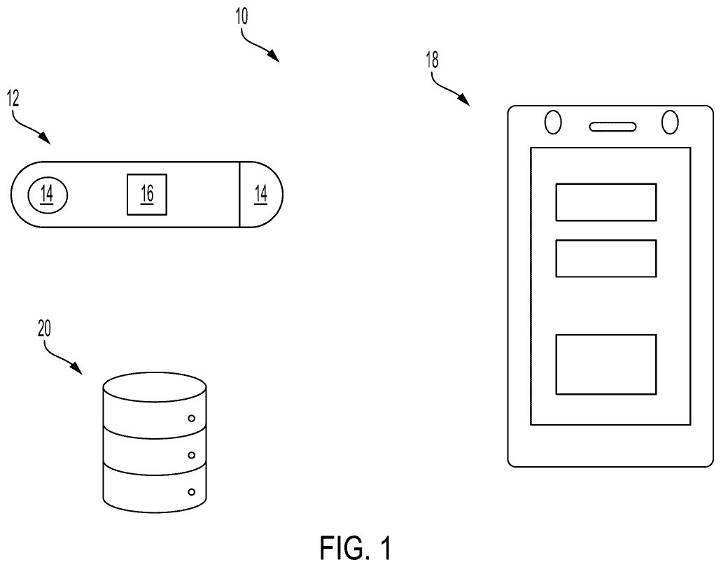

FIG. 1 shows a chemical sensing system 10 (hereinafter “the system 10” for brevity) with schematic representations of components that can be used to sense, measure, and monitor physiological parameters. In particular, components of the system 10 can ultimately be used to estimate analyte concentrations using an implantable medical device.

The system 10 includes an implantable medical device 12, which includes one or more electrodes 14 and a chemical sensor assembly 16. The electrodes 14 can comprise a conductive material and be configured to sense cardiac activation signals. Cardiac activation signals can be used to generate electrocardiogram (ECG) data. In some instances, the implantable medical device 12 does not include electrodes.

The chemical sensor assembly 16 can include a sensing element with a polymeric matrix permeable to analytes such as potassium, sodium, and/or creatinine. The sensing element can include an interior volume with various chemical indicators (e.g., beads or film for detecting an ion concentration of a bodily fluid when implanted in the body). Analytes can diffuse through an outer barrier layer and onto and/or into the chemical indicators where the analytes can bind with ion selective sensors to produce an optical response (e.g., a change in optical properties such as a change in concentration, a fluorimetric response, a colorimetric response). The optical response can be monitored and used to estimate analyte levels (e.g., analyte concentrations).

The system 10 can also include a computing device 18 such as a mobile computing device (e.g., a smart phone, a tablet, and the like) and/or a computing system 20 (e.g., a server). Estimated analyte levels can be used by the computing device 18 to monitor and evaluate a person's kidney and/or cardiac performance among other functions. In certain instances, the implantable medical device 12 itself is programmed to estimate analyte levels based on optical properties of the chemical sensor. Additionally or alternatively, the computing device 18 and/or computing system 20 is programmed to estimate analyte levels. The device 18 and/or the computing system 20 can communicate (e.g., wirelessly) with the implantable medical device 12 and each other.

### Implantable Medical Device

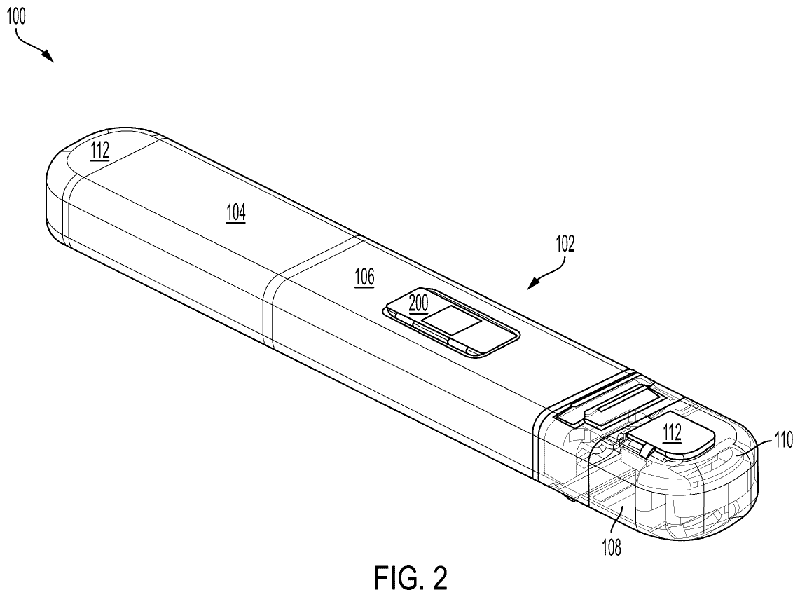

FIG. 2 shows an implantable medical device (IMD) 100 that includes a body 102 with various sections such as a battery module 104 (e.g., a section that houses a battery), an electronics housing 106 (e.g., a section that is hollow and houses various electronics such as a printed circuit board, integrated circuitry such as controllers and processors, and the like), a header 108 (e.g., a section that houses components such as an antenna 110), and electrodes 112 at opposite ends of the body 102. In certain instances, the IMD 100 is header-less.

The battery module 104 can include an electrochemical cell disposed therein to provide power for the IMD 100. The electrochemical cell can be a single-use cell (e.g., a primary lithium-based cell) or a rechargeable cell (e.g., a secondary lithium-ion-based call). Rechargeable cells can comprise a rechargeable lithium-based cell such as a lithium-manganese dioxide (Li anode/MnO2 cathode) battery, however, other primary lithium battery chemistries are also contemplated herein-including, but are not limited to, CFx, SVO, hybrid CFx/Mn02, hybrid CFx/SVO, and the like.

The electrodes 112 comprise a conductive material and are arranged to sense cardiac activation signals.

The IMD 100 also includes a chemical sensor assembly 200 (hereinafter “the chemical sensor 200” for brevity). The chemical sensor 200 can include one or more chemical indicators that are in communication (e.g., indirect communication) with a person's blood. For example, the indicators may be exposed to interstitial fluid, which is in communication with blood. As described further herein, the chemical indicators can change optical properties as analyte levels change. Estimating an analyte level using the chemical sensor 200 can include sensing one or more optical properties of the chemical sensor 200 and estimating an analyte level based on the optical property. In certain instances, estimating an analyte level occurs periodically (e.g., every 30 minutes, once an hour) or on demand (e.g., when a patient or physician initiates the comparison). Although the chemical sensors may react in real-time (e.g., the chemical indicators change optical properties in real-time as analyte levels change in real-time), transmission of or estimating an analyte level less often can save computing and battery resources and may be preferable because analyte levels may not change significantly minute-by-minute.

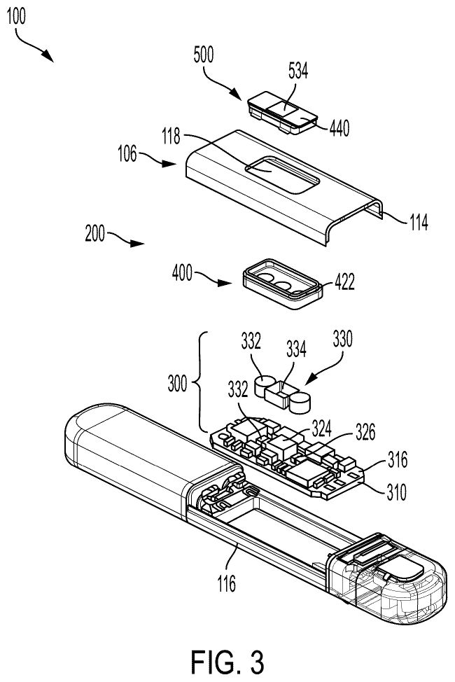

FIG. 3 shows an exploded view of the IMD 100 and, in particular, components of the chemical sensor 200. The chemical sensor 200 is described herein as including various subassemblies. As before, the IMD 100 includes an electronics housing 106 that is hollow and houses various electronics including at least some subassemblies of the chemical sensor 200. The electronics housing 106 includes a top housing shell 114 and a bottom housing shell 116. The top housing shell 114 and the bottom housing shell 116 can be formed of a biocompatible electrically conductive material such as, for example, titanium or a titanium alloy. In various instances, the top housing shell 114 and the bottom housing shell 116 can be attached together (e.g., by welding such as laser welding, by brazing, and the like) along intersecting edges thereof, such as the lateral edges thereof. The top housing shell 114 and the bottom housing shell 116 can define a space there between to hold various components, including subassemblies of the chemical sensor 200 herein. In some instances, self-aligning mechanisms (e.g., liners, fiducials, marks, and the like) can be positioned on a bottom surface of the bottom housing shell 116 to facilitate vertical stacking of the subassemblies with a desired tolerance. Other housing designs can be used with the chemical sensor 200. For example, the housing could comprise fewer separate sections that are made from materials such as ceramics, plastics, sapphire, etc.

In the instance depicted in FIG. 3, the subassemblies of the chemical sensor 200 include an opto-electronic assembly 300, an optical feedthrough 400, and a chemical sensor cassette 500. It is to be understood that the chemical sensor 200 can include other subassemblies or components which may be coupled to one or more of the opto-electronic assembly 300, the optical feedthrough 400, and the chemical sensor cassette 500.

FIG. 3 shows the opto-electronic assembly 300 including a circuit board 310, optical emitters 322 and 326 coupled to the circuit board 310, and an optical detector 324 coupled to the circuit board 310. The circuit board 302 can include a wide variety of substrates with metallic conductors. For example, the circuit board 302 can include a ceramic substrate, a silicon wafer substrate, or other types of substrates with metallic conductors. In certain instances the optical detector 324 is a photodiode and the optical emitters 322 and 326 are light emitting diodes (LEDs).

The opto-electronic assembly 300 further includes an optical seal 330. The optical seal 300 can have various designs. In one design, the optical seal 300 is shaped and positioned to at least partially surround the optical detector 324 (and not to surround the optical emitters 322 and 326). In another design, the optical seal 300 at least partially surrounds the optical emitters 322 and 326 but not the optical detector 324. In another design, the optical seal 330 (or multiple separate optical seals) surround both the optical emitters 322 and 326 and the optical emitter 324. The optical seal 330 is arranged to confine light emitted by the optical emitters 322 and 326 and reduce undesirable light leak paths when light transmits from the optical emitters 322 and 326 into the chemical sensor cassette 500. When surrounding the optical detector 324, the optical seal 330 reduce undesirable light leak paths out of or passed into the area within the optical seal 330. The optical seal 330 can be formed of a variety of materials. Examples includes an opaque elastic material such as, for example, a dark (e.g., black) rubber-based material, a dark silicone elastomer, a non-opaque base material that is coated with a reflective or absorptive material, etc. In some instances, the optical seal 330 is an O-ring or O-ring-like component. An optical fill material can be at least partially arranged within the optical seal 330. The optical fill material can be comprised of an optically transparent adhesive (e.g., acrylics, silicones, and the like) or optically clear adhesive.

The optical feedthrough 400 can fit into a sensor window 118 defined by the top housing shell 114. The optical feedthrough 400 creates a well 422. The chemical sensor cassette 500 can be at least partially positioned in the well 422. In some instances, the chemical sensor cassette 500 can be releasably attached to the optical feedthrough 400 and/or the top housing shell 114 such that the chemical sensor cassette 500 is exchangeable to allow easy switching of analytes and enable pre-calibration and testing without dissembling other components of the chemical sensor 200. A top cover 440 can be used for one or more functions such as a mechanical shield, a screen (e.g., coarse grid) for porosity, a matrix for stabilizing a bio interface material like fibers or hydrogel, and/or a retainer to confine the chemical sensor cassette 500 in the well 422.

Portions of the optical feedthrough 400 such as a ferrule can be formed of a biocompatible material such as, for example, titanium or a titanium alloy, and the material can be electrically conductive in certain instances. Optical windows in the optical feedthrough 400 can be formed of glass, crystal, ceramic, polymer, or the like, including, for example, quartz, silica, or sapphire. In various instances, the optical windows can be formed of a low-index glass, crystal, ceramic, or polymer, such as one having an index of refraction of 1.5 or less.

In various instances, the optical emitters 322 and 326 can be configured to emit light which is confined by the optical seal 330 and directed through the optical feedthrough 400 and into the chemical sensor cassette 500 disposed in the well 422. Such light can interface with a chemical indicator of the chemical sensor cassette 500, being scattered or transmitted, and can be directed downwards back through the optical feedthrough 400 to the optical detector 324.

### Chemical Sensor Cassette

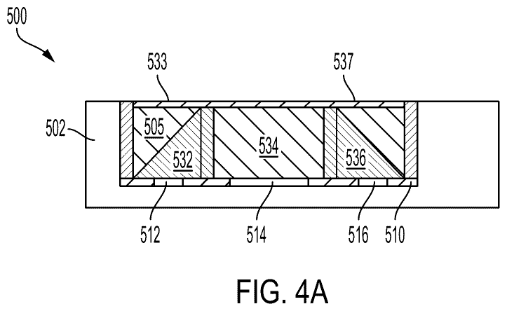

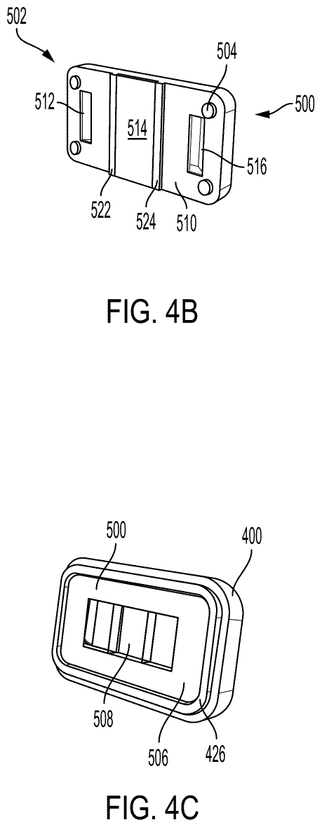

Referring to FIGS. 4A-4C, the chemical sensor cassette 500 includes a cassette housing 502 including an interior space 505. The cassette housing 502 includes a bottom portion 510. Three bottom optical apertures 512, 514, and 516 are formed through the bottom portion 510 although a different number of apertures can be formed. The bottom optical apertures 512, 514, and 516 can be respectively aligned with optical windows of the optical feedthrough 400 when the chemical sensor cassette 500 is positioned in the well 422 of the optical feedthrough 400.

As shown in FIG. 4A, the chemical sensor cassette 500 includes one or more chemical indicators 534 positioned in the interior space 505 and aligned with the bottom aperture 514. First reflector 532 and second reflector 536 are also positioned in the interior space 505 to direct light to or from the chemical indicators 534. It is to be understood that a different number of reflectors can be used with the chemical sensor cassette 500. The first reflector 532 and the second reflector 536 are positioned inside the respective optical chambers 533 and 537 and aligned with the respective bottom optical apertures 512 and 516. In some instances, the first reflector 532 is arranged to receive light from the optical windows of the optical feedthrough 400 and reflect light towards the chemical indicator 534. In some instances, the second reflector 536 is arranged for one of the following: (1) to receive light from the optical aperture 516 and reflect light towards the chemical indicator 534, or (2) to receive light reflected by the first reflector 532 and transmitted through the chemical indicator 534, and reflect the light towards the optical aperture 516.

As shown in FIG. 4B, in some instances, the optical apertures 512 and 516, which can be aligned with the corresponding optical emitters 322 and 326, each have a slot shape (e.g., a rectangular shape or elongated shape). The optical aperture 514, which can be aligned with the corresponding optical detector 324, is in the form of an optical window which can be relatively wider than the slot shape. Bars 522 and 524 are positioned between the optical apertures 512, 514 and 516 to prevent undesired crosstalk between the adjacent optical apertures/channels.

As shown in FIG. 4B, in some instances, the cassette housing 502 has standoff protrusions 504 positioned at or near each corner of the bottom portion 510 of the cassette housing 502. The standoff protrusions 504 are configured to contact the corresponding corners of the bottom surface of the well and control the spacing between the bottom portion 510 of the cassette housing 502 and the bottom surface of the well of the optical feedthrough 400.

As shown in FIG. 4C, the chemical sensor cassette 500 is at least partially positioned inside the optical feedthrough 400. A top window 508 is positioned on the top surface 506. The top window 508 is designed to permit desired analytes to be in communication with the chemical indicators. For example, analytes can diffuse through an outer barrier layer of the top window 508 and to the chemical indicators of the chemical sensor cassette 500. As the concentration of a given analyte changes, optical properties of the chemical indicators can change. In some instances, the top window 508 is formed of a polymeric material including, for example, poly(2-hydroxyethyl methacrylate) (pHEMA), polyvinyl alcohol (pVA), polyethylene glycol methacrylate/polyethylene glycol diacrylate (PEGMEA/PEGDA), polyethylene glycol/poly(vinylpyrrolidone (PEG/PVP), polyacrylonitrile (PAN), and mixtures, blends, and copolymers of the above polymers.

In some instances, the top surface 506 of the chemical sensor cassette 500 can include a mask coating. The mask coating can be disposed on the top surface 506. The mask coating can be an opaque material and reduce the amount of ambient light from entering the inside of the chemical sensor cassette 500 and to reduce undesirable light leak paths. In some instances, the mask coating can include carbon black and/or various pigments or components to render the coating opaque.

### Chemical Sensing Chemistry

In certain instances, color of the chemical indicator 534 comprises the sum of the absorption, transmission, reflectance, and fluorescence properties of the chemical indicator material. Put another way, the chemical indicator 534 can comprise a material that changes optical properties with changes in concentration of a given analyte—and such optical properties can be measured by analyzing an image of the chemical indicator 534.

The chemical indicator 534 may include a sensor material having various configurations or forms, for example, response elements or ion-selective sensor molecules encompassed in the form of beads or a film. The beads can include a polymer matrix (e.g., polymeric beads), porous glass material, or another type of porous or non-porous material. The beads can have various diameters which can be all the same or can be different, such as different between different response element types. In some instances, as will be discussed in more details below, the film can include multiple layers with varying thicknesses including, for example, a backing layer, a polymeric indicator layer including the ion-selective sensor molecules adjacent the backing layer, and/or a migration-limiting barrier layer adjacent the polymeric indicator layer configured to decrease leaching of the ion-selective sensor molecules from the polymeric indicator layer. Various chemistries described below can be disposed within and/or bonded to the polymer matrix or porous glass material of the beads, or the polymeric indicator layer of the film.

In certain instances, the chemical indicator 534 comprises ion-selective sensor molecules including a lipophilic indicator dye (e.g., a lipophilic fluorescent indicator dye or a lipophilic colorimetric indicator dye). Lipophilic indicator dyes can include, but are not limited to, ion selective sensors such as ionophores or fluorophores. In certain instances, ionophores can include sodium-specific ionophores, potassium-specific ionophores, calcium-specific ionophores, magnesium-specific ionophores, and lithium-specific ionophores. In certain instances, fluorophores can include lithium-specific fluorophores, sodium-specific fluorophores, and potassium-specific fluorophores.

In some instances, the chemical indicator 534 can include components (e.g., response elements or ion-selective sensor molecules) that are configured for a colorimetric response, a photoluminescent response, or another optical sensing modality. For example, the chemical indicator 534 can include an element that changes color based on binding with or otherwise complexing with a specific chemical analyte. As one specific example, creatinine reacts with a molecule which changes pH and color on the chemical indicator 534. In some instances, the chemical indicator 534 can include a complexing moiety and a colorimetric moiety. Those moieties can be a part of a single chemical compound (e.g., a non-carrier-based system) or can be separated on two or more different chemical compounds (e.g., a carrier-based system). The colorimetric moiety can exhibit differential light absorbance on binding of the complexing moiety to an analyte.

Some of the chemical indicators 534 may not require a separate compound to both complex an analyte of interest and produce an optical response. By way of example, in some instances, the response elements or ion-selective sensor molecules can include a non-carrier optical moiety or material wherein selective complexation with the analyte of interest directly produces either a colorimetric or fluorescent response. As an example, a fluoroionophore can be used and is a compound including both a fluorescent moiety and an ion complexing moiety. As merely one example, (6,7-[2.2.2]-cryptando-3-[2″-(5″-carboethoxy)thiophenyl]coumarin, a potassium ion selective fluoroionophore, can be used (and in some cases covalently attached to polymeric matrix or membrane) to produce a fluorescence-based K+ non-carrier response element. An exemplary class of fluoroionophores are the coumarocryptands. Coumarocryptands can include lithium specific fluoroionophores, sodium specific fluoroionophores, and potassium specific fluoroionophores. For example, lithium specific fluoroionophores can include (6,7-[2.1.1]-cryptando-3-[2″-(5″-carboethoxy)furyl]coumarin. Sodium specific fluoroionophores can include (6,7-[2.2.1]-cryptando-3-[2″-(5″-carboethoxy)furyl]coumarin. Potassium specific fluoroionophores can include (6,7-[2.2.2]-cryptando-3-[2″-(5″-carboethoxy)furyl]coumarin and (6,7-[2.2.2]-cryptando-3-[2″-(5″-carboethoxy)thiophenyl]coumarin.

Analytes detected herein can include, but are not limited to, potassium, sodium, calcium, blood urea nitrogen (BUN), creatinine, and the like.

### Sensor Configuration I—Beads Surrounded by Hydrogel

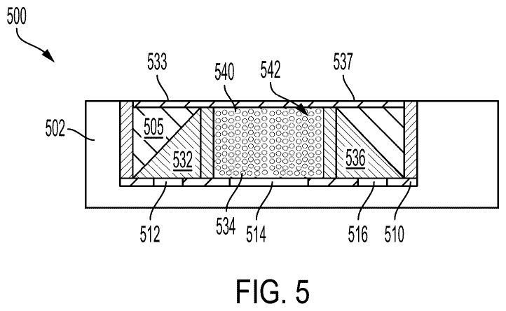

In certain instances, for example as shown in FIG. 5, the chemical indicator 534 comprises a plurality of indicator beads 540. The beads 540 can include a polymer matrix (e.g., polymeric beads), porous glass material, or another type of porous or non-porous material. In some instances, for example as shown, the beads 540 can have various diameters which can be all the same. In yet some instances, the beads 540 can have various diameters which can be different. The diameter of the beads 540 may range from about 1 to about 20 micrometers. In some instances, the indicator beads 540 are polymeric beads having embedded therein ion-selective sensor molecules. In certain instances, the indicator beads 540 are polymeric beads including a polymer matrix, for example, poly(vinyl) chloride (PVC), crosslinked PVC, polyimide, silicone rubber, polyurethane, acrylate, perfluoropolymers, or any polymer known and used in the art for ion-selective membrane materials. Physiological analytes of interest can diffuse into and out of the indicator beads 540 and bind with an ion-selective sensor molecule to result in a fluorimetric or colorimetric response.

In certain instances, the chemical indicator 534 comprises a hydrogel 542 surrounding the indicator beads 540. Hydrogel 542 may be a three-dimensional, crosslinked polymeric structure that is substantially insoluble in water, but which is capable of absorbing and retaining water (e.g. large quantities of water) to form a substantially stable structure. In certain instances, water can penetrate in between polymer chains of a polymer network, subsequently causing swelling and the formation of the hydrogel 542. In certain instances, hydrogel 542 is super-absorbent (e.g., containing more than about 90% water). In some instances, hydrogel 542 can contain greater than about 91%, or about 92%, or about 93%, or about 94%, or about 95%, or about 96%, or about 97%, or about 98%, or about 99%, and for each of the foregoing, less than 100% of water, or within any range encompassing any two of these values as endpoints.

In some instances, the hydrogel 542 comprises a hydrophilic polymer bonded to a crosslinked network, and the crosslinked network is formed by crosslinking a hydrophilic polymer (e.g., functionalized PEG) with a crosslinking agent or a crosslinker (e.g., acrylamide, activated ester, amine, functionalized PEG). In certain instances, the crosslinked network is a polyacrylate network. In certain instances, the crosslinked network is a polyacrylamide network.

In some instances, the hydrogel 542 may be a homopolymer hydrogel comprising a polymer network derived from a single species of monomer. In some instances, the hydrogel 542 may be a copolymeric hydrogel comprised of two or more different monomer species with at least one hydrophilic component. In some instances, the hydrogel 542 may be a multipolymer Interpenetrating polymeric network (IPN) hydrogel made of two independent crosslinked synthetic and/or natural polymer components, contained in a network form.

In some instances, the hydrogel 542 comprises a hydrogel network with a hydrophilic backbone and functionalized end-group(s) that can be polymerized into the hydrogel network. In some instances, the hydrogel 542 may be an IPN hydrogel having multiple polymeric networks interpenetrating with one another, the interpenetrating polymeric networks include a star PEG polymer having more than two arms and one or more additional components such as a polyacrylate network, a polyacrylamide network, a polycarbonate network, a polyurea network, and/or a polyurethane network.

### Hydrogel Polymer

In certain instances, the hydrogel 542 is formed by polymerization and parallel crosslinking of multifunctional monomers, or multiple-step procedures involving synthesis of polymer molecules having reactive groups and their subsequent crosslinking. Hydrogel-forming natural polymers include proteins such as collagen and gelatin and polysaccharides such as starch, alginate, and agarose. Synthetic polymers that form the hydrogel 542 may be prepared using chemical polymerization methods. Synthetic hydrogel polymers may include monomers such as acrylic acid (AA), acrylamide (AM), and polymers such as polyvinyl alcohol (PVA), polyethylene glycol (PEG), polyethylene oxide (PEO), poly(2-hydroxyethyl methacrylate) (PHEMA), 2-hydrocyethyl methacrylate (HEMA), polyacrylic acid (PAA), and polyacrylamide (PAAm). The hydrogel 542 may include natural or synthetic polymers or combinations of both. In certain instances, the polymers suitable for polymerization/crosslinking to form the hydrogel 542 are hydrophilic, which means they are substantially miscible with water. In some instances, hydrogel 542 comprises a synthetic hydrophilic polymer including, for example, polyethylene glycol (PEG).

Polymers suitable for crosslinking and forming the hydrogel 542 may include functionalized PEG including, for example, mono-functional PEGs, bi-functional PEGs, and multi-functional PEGs, for example, functionalized star PEGs having more than two arms (e.g., 3-20 arms).

The bi-functional and multi-functional PEGs as used herein for forming the hydrogel 542 may have an average molecular weight ranging from, for example, from about 100 Da to about 120,000 Da. In some instances, the bi-functional and multi-functional PEGs have an average molecular weight ranging from about 100 Da to about 150 Da, from about 150 Da to about 200 Da, from about 200 Da to about 250 Da, from about 250 Da to about 300 Da, from about 300 Da to about 500 Da, from about 500 Da to about 750 Da, from about 750 Da to about 1,000 Da, from about 1,000 Da to about 2,000 Da, from about 2,000 Da to about 5,000 Da, from about 5,000 Da to about 10,000 Da, from about 10,000 Da to about 15,000 Da, from about 15,000 Da to about 20,000 Da, from about 20,000 Da to about 30,000 Da, from about 30,000 Da to about 40,000 Da, from about 40,000 Da to about 50,000 Da, from about 50,000 Da to about 60,000 Da, from about 60,000 Da to about 70,000 Da, from about 70,000 Da to about 80,000 Da, from about 80,000 Da to about 90,000 Da, from about 90,000 Da to about 100,000 Da, from about 100,000 Da to about 110,000 Da, from about 110,000 Da to about 120,000 Da, or within any range encompassing any two of these values as endpoints.

In its most common form, PEG is a linear molecule containing free hydroxyl groups at each terminus according to Formula (I) as follows:

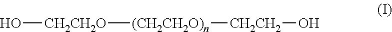

where n is from about 8 to about 4,000. A functionalized PEG as used herein is a PEG molecule where at least one of the hydroxyl end groups in Formula (I) has been replaced with a reactive functional group, for example an amine (—NH₂), thiol (—SH), carboxyl (—COOH), ester (—CO—OR′), alkyne (—C═C), or amide (—C═O), allowing for further attachment to other molecules through chemical reactions.

Mono-functional PEGs as describe herein refer to PEG molecules, in which one of the terminal functional groups is capped with an essentially inactive group, resulting in only one functional group remaining. A mono-functional PEG has a structure according to Formula (II) as follows:

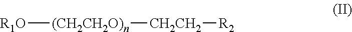

where R₁ is a capping group such as hydroxyl functional group (—OH), a methoxy group, an ethoxy group, or an n-propoxy group, and R² is a reactive functional group including, for example, an acrylamide group (—NH—CO—CH₂═CH₂), an amine (—NH₂), an ester (—CO—OR′), or a thiol (—SH).

An exemplary mono-functional PEG that may be used for forming hydrogel 542 may have the below structure (P1), where the functional group R₂ is an acrylamide:

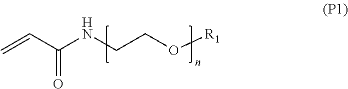

Bi-functional PEGs as describe herein refer to PEG molecules having two terminal functional groups that may be the same or may be different. A bi-functional PEG has a structure according to Formula (III) as follows:

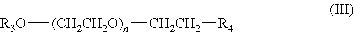

where R₃ and R₄ are functional groups including, for example, a hydroxyl functional group (—OH), an acrylamide group (—NH—CO—CH₂═CH₂), an amine (—NH₂), an ester (—CO—OR′), or a thiol (—SH). When the two end groups R₃ and R₄ are the same, the bi-functional PEG may be referred to as a homo-bifunctional PEG. When the wo end groups R₃ and R₄ are different, the bi-functional PEG may be referred to as a hetero-bifunctional PEG.

An exemplary bi-functional PEG that may be used for forming hydrogel 542 may have the below structure (P2), where both of the functional groups R₃ and R₄ are acrylamides:

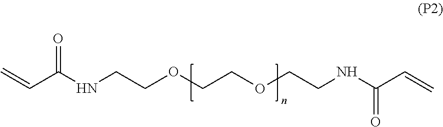

where R₁ is a capping group such as methoxy, ethoxy, and n-propoxy.

Another exemplary bi-functional PEG that may be used for forming hydrogel 542 may have the below structure (P3), where both of the functional groups R₃ and R₄ are amines:

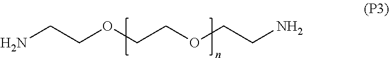

Another exemplary bi-functional PEG that may be used for forming hydrogel 542 may have the below structure (P4), where both of the functional groups R₃ and R₄ are activated esters, such as, but not limited to, succinimidyl esters:

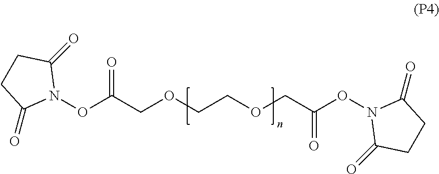

Multi-functional PEGs as describe herein refer to non-linear PEG molecules having more than two arms (e.g., three or more), and at least one functional group at a terminus. Multi-functional PEGs may also be referred to as functionalized star PEGs having more than two arms (e.g., 3-20 arms). The multi-functional PEGs as used herein for forming hydrogel 542 may have an average molecular weight ranging from, for example, about 300 Da to about 120,000 Da. In some instances, the multi-functional PEGs have an average molecular weight ranging from about 300 Da to about 400 Da, from about 400 Da to about 500 Da, from about 500 Da to about 750 Da, from about 750 Da to about 1,000 Da, from about 1,000 Da to about 2,000 Da, from about 2,000 Da to about 5,000 Da, from about 5,000 Da to about 10,000 Da, from about 10,000 Da to about 15,000 Da, from about 15,000 Da to about 20,000 Da, from about 20,000 Da to about 30,000 Da, from about 30,000 Da to about 40,000 Da, from about 40,000 Da to about 50,000 Da, from about 50,000 Da to about 60,000 Da, from about 60,000 Da to about 70,000 Da, from about 70,000 Da to about 80,000 Da, from about 80,000 Da to about 90,000 Da, from about 90,000 Da to about 100,000 Da, from about 100,000 Da to about 110,000 Da, from about 110,000 Da to about 120,000 Da, or within any range encompassing any two of these values as endpoints.

A multi-functional PEG having three arms that may be used for forming hydrogel 542 may have the below structure (P5):

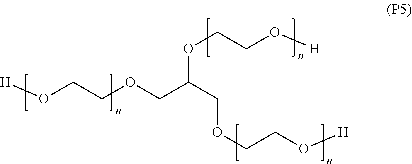

A multi-functional PEG having four arms that may be used for forming hydrogel 542 may have the below structure (P6):

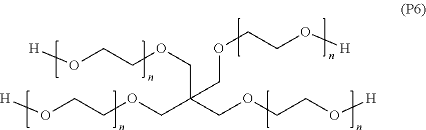

A multi-functional PEG having six arms that may be used for forming hydrogel 542 may have the below structure (P7):

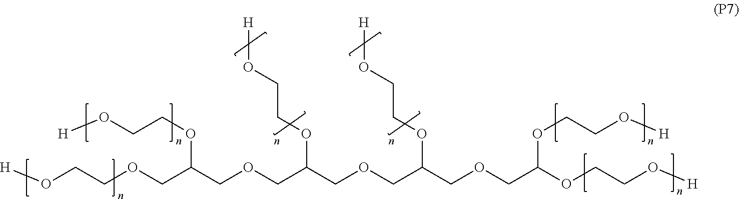

### Crosslinking Agent

A crosslinker (or crosslinking agent) refers to a molecule that may form a three-dimensional crosslinked network when reacted with the appropriate base monomers or polymers (e.g., hydrogel polymers as described above). A crosslinker can be any molecule that is suitable for connecting the base monomers or polymers via crosslinks to form the polymer network and thus the hydrogel 542. Crosslinking agents generally have two or more reactive functional groups at different sites of the molecule. Typically, these sites contain polymerizable ethylenic unsaturation groups. During curing, they form a covalent bond with two different polymer chains and form a stable three-dimensional network to improve the strength of the polymer.

Chemical properties of the hydrogel 542 may be attained by incorporating specific polymers, co-monomers, and crosslinkers and by changing the crosslinking degree. A strong hydrogel network may be obtained with increasing the degree of crosslinking. Crosslinking at high amounts may result in low elongation and elasticity with greater brittleness. An optimal degree of crosslinking for hydrogels is useful in order to retain the compromise between mechanical strength and elasticity.

Crosslinkers suitable for crosslinking the monomers/polymers and forming the hydrogel 542 may include, for example, an acrylamide (e.g., a mono-functional acrylamide monomer and/or a bi-functional acrylamide monomer), an isocyanate (e.g., diisocyanate), a multi-arm activated crosslinker (e.g., an activated ester, an amine), and/or the functionalized PEGs as described above that may be used as both the base polymer and a crosslinker. Other suitable crosslinkers may be used by those skilled in the art, and may include for example, ethylene glycol dimethacrylate, trimethyloylpropane trimethacrylate, diethyleneglycol dimethacrylate, bisphenol A dimethacrylate, diglycidyl bisphenol A dimethacrylate, dimethacrylate-terminated polyethylene glycol and reactive linear polyether modified silicones, carbonates (e.g., to form a polycarbonate network), ureas (e.g., to form a polyurea network), urethanes (e.g., to form a polyurethane network), etc.

In some instances, the crosslinker used for forming hydrogel 542 may include mono-functional acrylamide monomers including but not limited to the below structures (C1)-(C9):

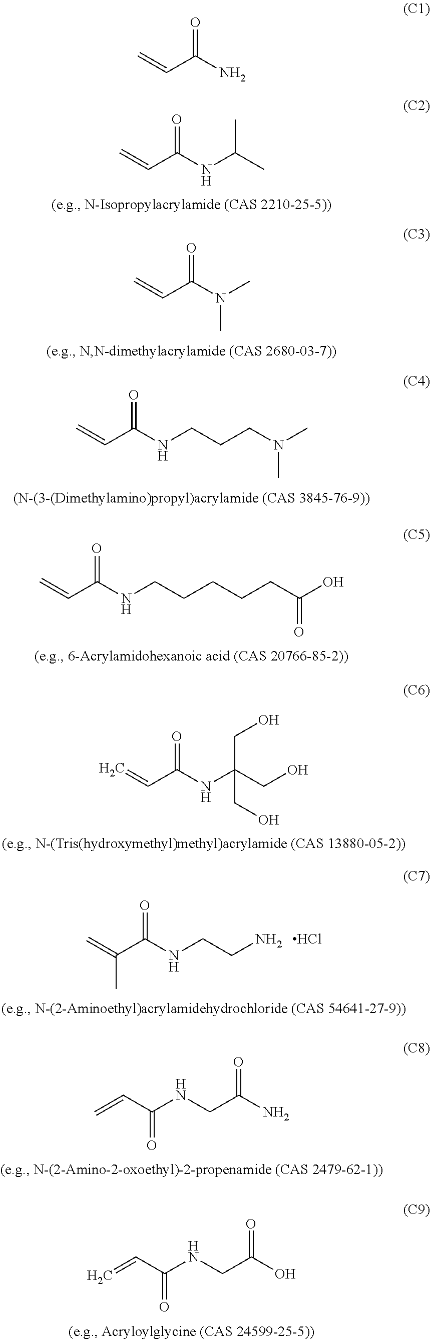

In some instances, the crosslinker used for forming hydrogel 542 may include mono-functional acrylamide monomers including but not limited to the below structures (C10)-(C18):

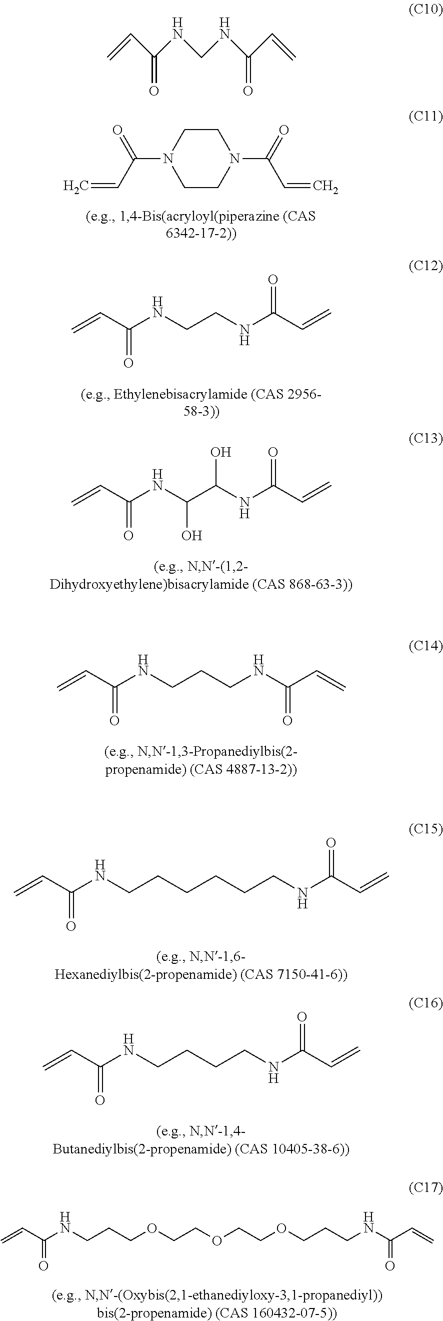

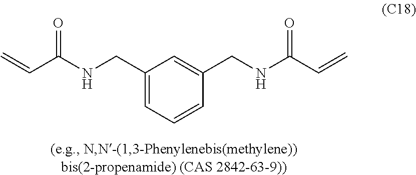 [SMILES: C=CC(=O)NCc1cccc(CNC(=O)C=C)c1]

In certain instances, the crosslinker used for forming hydrogel 542 may include an isocyanate (C19) including but not limited to a diisocyanate having the below structure (C20):

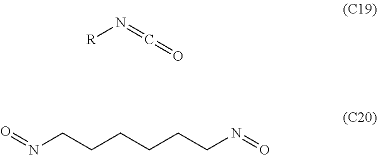

In certain instances, the crosslinker used for forming hydrogel 542 may include a multi-arm activated crosslinker including but not limited to an activated ester and/or an amine having the below structures (C21)-(C22):

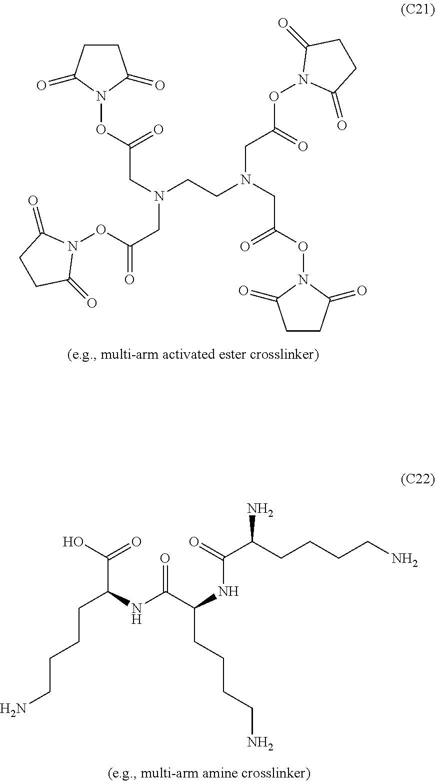 [SMILES: O=C(CN(CCN(CC(=O)ON1C(=O)CCC1=O)CC(=O)ON1C(=O)CCC1=O)CC(=O)ON1C(=O)CCC1=O)ON1C(=O)CCC1=O ; NCCCC[C@H](NC(=O)[C@H](CCCCN)NC(=O)[C@@H](N)CCCCN)C(=O)O]

In certain instances, the crosslinker used for forming hydrogel 542 may include functionalized PEGs that may be used as both the base polymer and a crosslinker, and may have a structure the same as, but not limited to, for example, structures (P1)-(P7) as described above.

In certain instances, the crosslinker used for forming the hydrogel 542 may include hydrolytically stable linkers that are suitable for crosslinking the polymers as described herein, for example, carbonate crosslinkers, urea crosslinkers, or any other crosslinkers known and used by a person of skills in the art.

### Hydrogel

As discussed above, the hydrogel 542 may be formed by polymerization of a base monomer or polymer with a crosslinker. Different types of polymerization include free radical polymerization, anionic or cationic polymerization, chain-growth or addition polymerization, condensation polymerization, ring-opening polymerization etc. The polymerization may be initiated by certain initiators, by light and/or heat, and may be mediated by catalysts. Suitable monomer, polymer, and crosslinkers for use to form the hydrogel 542 for use in surrounding and holding the indicator beads 540 are discussed above.

Chemical properties of hydrogel 542 may be attained by incorporating specific polymers, monomers, and crosslinkers and by changing the crosslinking degree. A strong hydrogel network may be obtained with increasing the degree of crosslinking. Crosslinking at high amounts may result in low elongation and elasticity with greater brittleness. An optimal degree of crosslinking for hydrogel 542 is useful in order to retain the compromise between mechanical strength and elasticity, and for retaining the indicator beads 540.

According to some instances, the hydrogel 542 may include from about 0.1% to about 10% of a crosslinker. In some instances, the hydrogel 542 may include from about 0.1% to about 0.5%, or from about 0.5% to about 1%, or from about 1% to about 1.5%, or from about 1.5% to about 2%, or from about 2% to about 2.5%, or from about 2.5% to about 3%, or from about 3% to about 3.5%, or from about 3.5% to about 4%, or from about 4% to about 4.5%, or from about 4.5% to about 5%, or from about 5% to about 5.5%, or from about 5.5% to about 6%, or from about 6% to about 6.5%, or from about 6.5% to about 7%, or from about 7% to about 7.5%, or from about 7.5% to about 8%, or from about 8% to about 8.5%, or from about 8.5% to about 9%, or from about 9% to about 9.5%, or from about 9.5% to about 10% of a crosslinker, or within any range encompassing any two of these values as endpoints. The percentage of crosslinker may be expressed as weight to volume, volume to volume, or weight to weight.

According to certain instances, the pore size of the hydrogel is tuned by varying the ratio of the concentration of polymer to the concentration of crosslinker. In some examples, the ratio of polymer to crosslinker is about 30:1, about 25:1, about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:30, or a ratio within any range encompassing any two of these values as endpoints. The ratio may be expressed as weight to volume, volume to volume, or weight to weight. In some instances, the hydrogel 542 may include multiple polymers each having a predetermined ratio of concentration of polymer to the concentration of crosslinker.

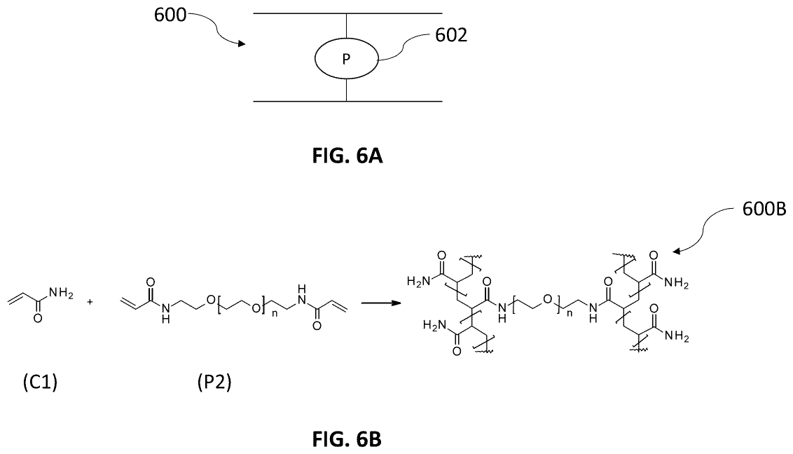

In certain instances, for example referring to FIGS. 6A-6B, without wishing to be bound by theory, hydrogel 542 may include a crosslinked network 600 having a polymer 602 connected to the network 600 via two bonds as shown in FIG. 6A. The crosslinked network 600 may be a polyacrylamide network 600B formed by crosslinking a bi-functional PEG (P2) with a mono-functional acrylamide crosslinker (C1), for example, as shown in FIG. 6B.

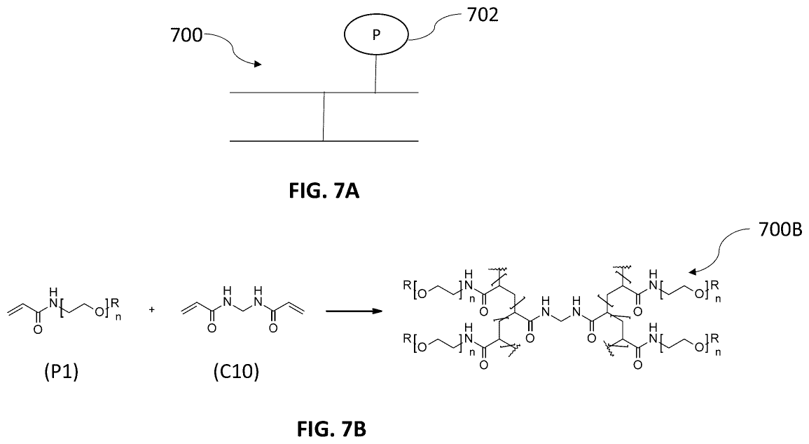

In certain instances, for example referring to FIGS. 7A-7B, without wishing to be bound by theory, hydrogel 542 may include a crosslinked network 700 having a polymer 702 connected to the network 700 via a single bond as shown in FIG. 7A. The crosslinked network 700 may be a polyacrylamide network 700B formed by crosslinking a mono-functional PEG (P1) with a bi-functional acrylamide crosslinker (C10), for example, as shown in FIG. 7B.

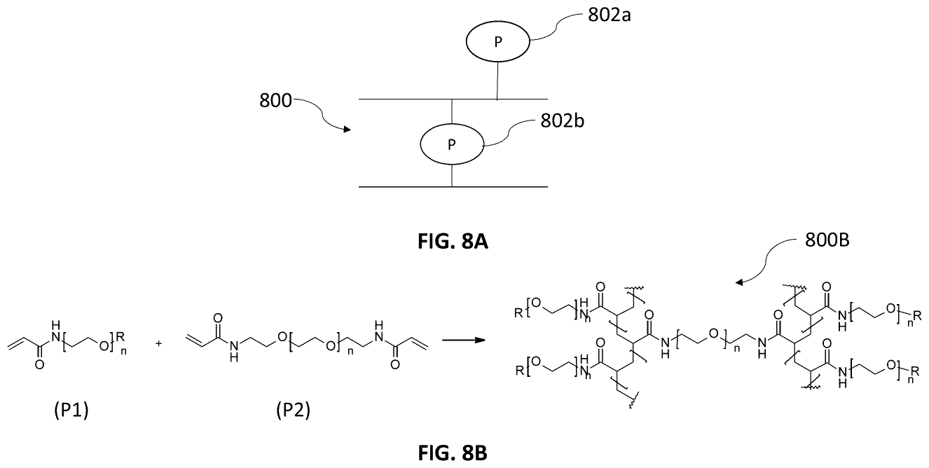

In certain instances, for example referring to FIGS. 8A-8B, without wishing to be bound by theory, hydrogel 542 may include a crosslinked network 800 having a polymer 802a connected to the network 800 via a single bond, and a polymer 802b connected to the network 800 via two bonds, as shown in FIG. 8A. The crosslinked network 800 may be a polyacrylamide network 800B formed by crosslinking a mono-functional PEG (P1) with a bi-functional PEG (P2), for example, as shown in FIG. 8B.

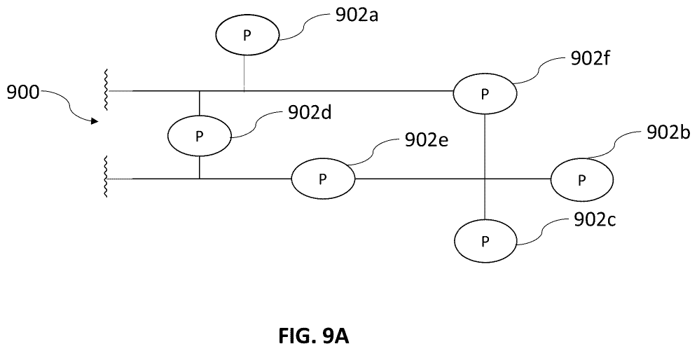

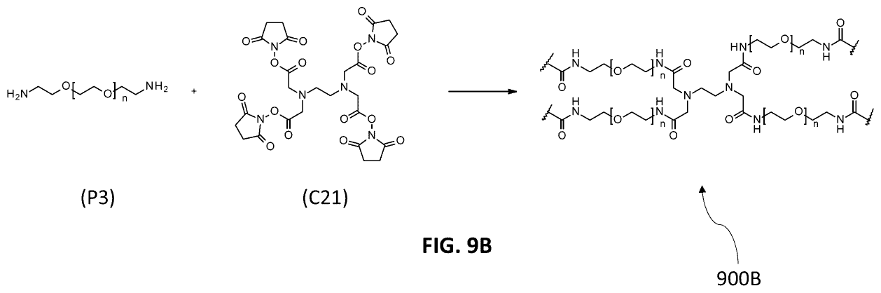

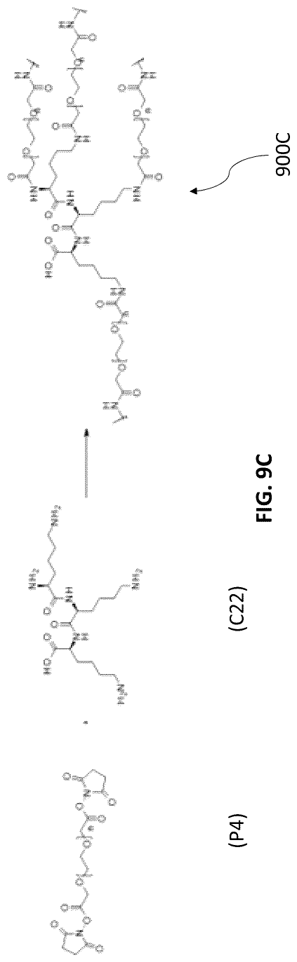

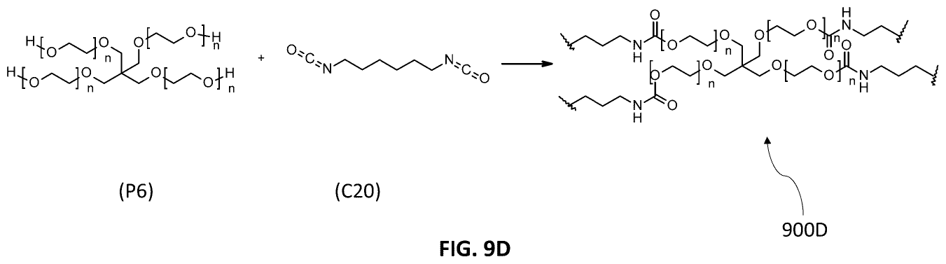

In certain instances, for example referring to FIGS. 9A-9D, without wishing to be bound by theory, hydrogel 542 may include a crosslinked network 900 having polymers 902a, 902b, and 903c connected to the network 900 via a single bond, and polymers 902d, 902e, and 902f connected to the network 900 via two bonds, as shown in FIG. 9A. In some instances, the crosslinked network 900 may be a polyamide network. In certain instances, the crosslinked network 900 may be a polyamide hydrogel. In some instances, for example, the crosslinked network 900 may be a polyamide (e.g., polyacrylamide) network 900B formed by crosslinking a bi-functional PEG (P3) with a multi-arm activated ester crosslinker (C21) as shown in FIG. 9B. In some instances, the crosslinked network 900 may be a polyacrylamide network 900C formed by crosslinking a bi-functional PEG (P4) with a multi-arm amine crosslinker (C22), for example, as shown in FIG. 9C. In some instances, the crosslinked network 900 may be a polyacrylamide network 900D formed by crosslinking a multi-arm functional PEG (P6) with a diisocyanate crosslinker (C20), for example, as shown in FIG. 9D.

In certain instances, hydrogel 542 may be an IPN hydrogel having multiple polymeric networks interpenetrating with one another, the interpenetrating polymeric networks include a star PEG polymer having more than two arms and one or more additional components such as an polyacrylate network, a polyacrylamide network, a polyurea network, and/or a polyurethane network. The star PEG polymer interpenetrated in the IPN includes more than two arms, for examples, 3 arms, or 4 arms, or 5 arms, or 6 arms, or 7 arms, or 8 arms, or 9 arms, or 10 arms, or 11 arms, or 12 arms, or 13 arms, or 14 arms, or 15 arms, or 16 arms, or 17 arms, or 18 arms, or 19 arms, or 20 arms.

### Sensor Configuration II—Film

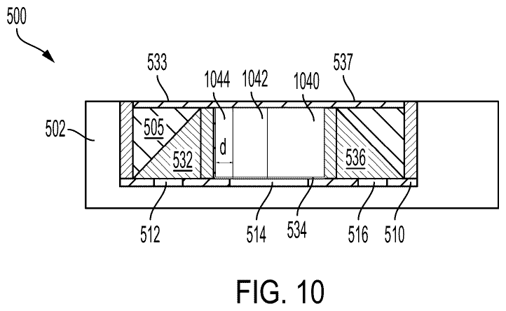

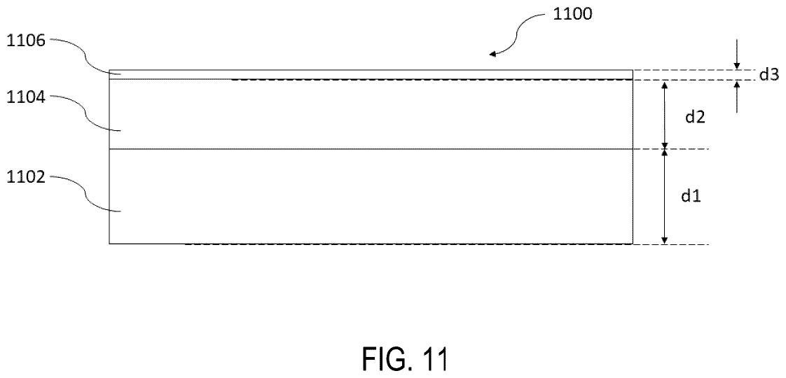

As discuss above with regard to FIG. 5, the chemical indicator 534 may include a sensor material having various configurations or forms, for example, response elements or ion-selective sensor molecules encompassed in the form of beads or a film. In certain instances, for example as shown in FIG. 10, the chemical indicator 534 includes a plurality of layers, and is different from the sensor configuration encompassing indicator beads as depicted in FIG. 5. The plurality of layers may include, for example, a backing layer 1040, an indicator layer 1042 adjacent the backing layer 1040, and a migration-limiting barrier layer 1044 adjacent the indicator layer 1042.

The migration-limiting barrier layer 1044 may be a hydrogel layer including any of the hydrogel as described above. Alternatively, the migration-limiting barrier layer 1044 may be a polymeric layer as discussed further in detail below. In some instances, the migration-limiting barrier layer 1044 including a hydrogel has a thickness “d” of from about 5 micrometers (μm) to about 1000 μm. In certain instances, the hydrogel layer 1044 has a thickness of from about 5 μm to about 10 μm, or from about 10 μm to about 20 μm, or from about 20 μm to about 50 μm, or from about 50 μm to about 100 μm, or from about 100 μm to about 150 μm, or from about 150 μm to about 200 μm, or from about 200 μm to about 300 μm, or from about 300 μm to about 500 μm, or from about 500 μm to about 750 μm, or from about 750 μm to about 1000 μm, or within any range encompassing any two of these values as endpoints. In some instances, the thickness of the migration-limiting barrier layer 1044 is less than or equal to about 1000 μm. In certain instances, the migration-limiting barrier layer 1106 is configured to decrease leaching of the ion-selective sensor molecules from the polymeric indicator layer without significantly increasing sensor response time.

The backing layer 1040, indicator layer 1042, and barrier layer 1044 may be collectively referred to as a “film-based sensor”. It is to be understood that FIG. 10 merely depicts one configuration of a chemical sensor cassette 500 suitable for housing and using with the film-based sensor. The film-based sensor may be housed in a cassette having a different configuration from the cassette 500 as depicted. Moreover, the film-based sensor may be used in any device for estimating analyte levels within a patient's body, and is not limited to the specific design of the IMD 100 or chemical sensor 200 as described herein.

### Film-Based Sensor

Referring to FIG. 11, a schematic cross-sectional view of a film-based sensor 1100 is shown in accordance with various instances herein. The film-based sensor may be used in any device for estimating analyte levels within a patient's body, and is not limited to the specific design of the IMD 100, or chemical sensor 200, or cassette 500 as described herein.

As shown, the film-based sensor 1100 includes a backing layer 1102, an indicator layer 1104 adjacent the backing layer 1102, and a migration-limiting barrier layer 1106 adjacent the indicator layer 1104. In some instances, the indicator layer 1104 is a polymeric indicator layer including ion-selective sensor molecules and a polymer. In certain instances, the migration-limiting barrier layer 1106 is configured to decrease leaching of the ion-selective sensor molecules from the polymeric indicator layer without significantly increasing sensor response time.

The backing layer 1102 may be made of any colorless material with desired flexibility. In some instances, the backing layer 1102 may be formed with a colorless polymer including for example, poly(ethylene terephthalate) (PET), polycarbonate (PC), poly(ethylene naphthalate) (PEN), poly(ether ether ketone) (PEEK), and/or poly(ether sulfone) (PES). In certain instances, the backing layer 1102 is formed of a polymer different from the polymer included in the indicator layer 1104. In certain instances, the backing layer 1102 is formed of PET.

The backing layer 1102 may have a thickness (d1) of from about 10 micrometers (μm) to about 150 micrometers (μm). In some instances, the backing layer 1102 has a thickness of from about 10 μm to about 20 μm, or from about 20 μm to about 30 μm, or from about 30 μm to about 40 μm, or from about 40 μm to about 50 μm, or from about 50 μm to about 60 μm, or from about 60 μm to about 70 μm, or from about 70 μm to about 80 μm, or from about 80 μm to about 100 μm, or from about 100 μm to about 120 μm, or from about 120 μm to about 150 μm, or within any range encompassing any two of these values as endpoints.

In some instances, the indicator layer 1104 includes ion-selective sensor molecules and a polymer. The ion-selective sensor molecules may be similar to the ones used in the chemical indicator 534 as described above, for example, lipophilic indicator dyes such as ionophores or fluorophores, or components configured for a colorimetric response, a photoluminescent response, or another optical sensing modality, or a non-carrier optical moiety or material wherein selective complexation with the analyte of interest directly produces either a colorimetric or fluorescent response. The polymer included in the indicator layer 1104 may be a different polymer than the one forming the backing layer 1102. In some instances, the polymer in the indicator layer 1104 includes polyvinyl chloride (PVC), crosslinked PVC, polyimide, silicone rubber, polyurethane, acrylate, perfluoropolymers, or any polymer known and used in the art for ion-selective membrane materials. In certain instances, the indicator layer 1104 further includes a plasticizer for plasticizing the polymer. In some instances, the plasticizer included in the indicator layer 1104 may be dioctyl phthalate (DOP), di-n-butyl phthalate (DBPH), dibutyl sebacate (DBS), o-nitrophenyl octyl phthalate, dioctyl sebacate (DOS), tricresylphosphate (TCP), dibutyl phthalate (DBP), nitrophenyl octyl phthalate, di-n-butyl phthalate (DBPH), dibutyl phosphate, tri-n-butyl phosphate (TBP), bis(1-butylpentyl) adipate, bis(2-ethylhexyl) phthalate, tris(2-ethylhexyl) phosphate (TEHP), tricresyl phosphate (TCP), o-nitrophenyl octyl ether (NPOE), dinonyladipinate (DNA), bis (2-ethylhexyl) sebacate, bis(2-ethylhexyl) adipate (DOA), and bis(2-ethylhexyl sebacate) (BEHS), or any plasticizer known and used in the art for plasticizing polymers as disclosed herein.

The indicator layer 1104 may have a thickness (d2) of from about 5 micrometers (μm) to about 50 micrometers (μm). In some instances, the indicator layer 1104 has a thickness of from about 5 μm to about 10 μm, or from about 10 μm to about 15 μm, or from about 15 μm to about 20 μm, or from about 20 μm to about 25 μm, or from about 25 μm to about 30 μm, or from about 30 μm to about 35 μm, or from about 35 μm to about 40 μm, or from about 40 μm to about 45 μm, or from about 45 μm to about 50 μm, or within any range encompassing any two of these values as endpoints.

As shown, the film-based sensor 1100 includes a migration-limiting barrier layer 1106 adjacent the indicator layer 1104. In certain instances, the migration-limiting barrier layer 1106 does not include the ion-selective sensor molecules. In some instances, the migration-limiting barrier layer 1106 is a selective barrier film that allows passage of analytes (e.g., calcium, potassium, sodium, etc.) to pass through the barrier film while preventing the ion-selective sensor molecules from leaching (e.g., migrating) out of the indicator layer 1104.

In some instances, the migration-limiting barrier layer 1106 includes the same polymer as the indicator layer 1104. In some instances, the migration-limiting barrier layer 1106 includes a different polymer than the indicator layer 1104. The polymers included in the migration-limiting barrier layer 1106 and the indicator layer 1104 may include, for example, poly(vinyl) chloride (PVC), crosslinked PVC, polyimide, silicone rubber, polyurethane, acrylate, perfluoropolymers, or any polymer known and used in the art for ion-selective membrane materials. In some instances, both the migration-limiting barrier layer 1106 and the indicator layer 1104 include polyvinyl chloride (PVC). In some instances, both the migration-limiting barrier layer 1106 and the indicator layer 1104 include crosslinked PVC. In certain instances, the indicator layer 1104 includes PVC and the migration-limiting barrier layer 1106 includes crosslinked PVC. In certain instances, the indicator layer 1104 includes crosslinked PVC and the migration-limiting barrier layer 1106 includes PVC.

The barrier layer 1106 may have a thickness (d3) of less than the thickness (d2) of the indicator layer 1104. In some instances, the barrier layer 1106 has a thickness of less than about 5 μm, or less than about 4.5 μm, or less than about 4 μm, or less than about 3.5 μm, or less than about 3 μm, or less than about 2.5 μm, or less than about 2 μm, or less than about 1.5 μm, or less than about 1 μm, or less than about 0.5 μm, or within any range encompassing any two of these values as endpoints.

In some instances, the collective thickness of the film-based sensor 1100 (d1+d2+d3) may be from about 5 micrometers (μm) to about 1000 micrometers (μm). In certain instances, the film-based sensor 1100 has a thickness of from about 5 μm to about 10 μm, or from about 10 μm to about 20 μm, or from about 20 μm to about 50 μm, or from about 50 μm to about 100 μm, or from about 100 μm to about 150 μm, or from about 150 μm to about 200 μm, or from about 200 μm to about 300 μm, or from about 300 μm to about 500 μm, or from about 500 μm to about 750 μm, or from about 750 μm to about 1000 μm, or within any range encompassing any two of these values as endpoints. In some instances, the thickness of the hydrogel layer 1044 is less than or equal to about 1000 μm to avoid significantly increasing sensor response time.

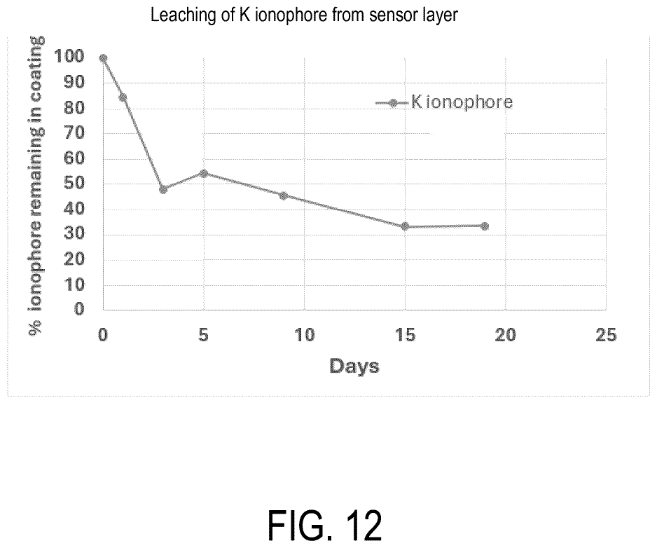

FIG. 12 is a graph showing leaching of ion-selective sensor molecules (e.g., potassium ionophore) from the indicator layer 1104 of a film-based sensor without a barrier film (e.g., barrier layer 1106). As shown, the percentage (%) of sensor molecules steadily decrease over the course of 15 days. For potassium ionophore, the % molecule remaining on day 5 is about 50%, on day 10 is less than 50%, and on day 15 is about 30%. Therefore, without a barrier film and as shown in FIG. 12 components such as the ion-selective sensor molecules leach (e.g., migrate) out of the indicator layer 1104 relatively quickly over the course of 5 to 15 days. According to instances of the present disclosure, the addition of a barrier film (e.g., barrier layer 1106) on top of the indicator layer 1104 prevents or minimizes leaching of components from the indicator layer 1104 without significantly increasing sensor response time.

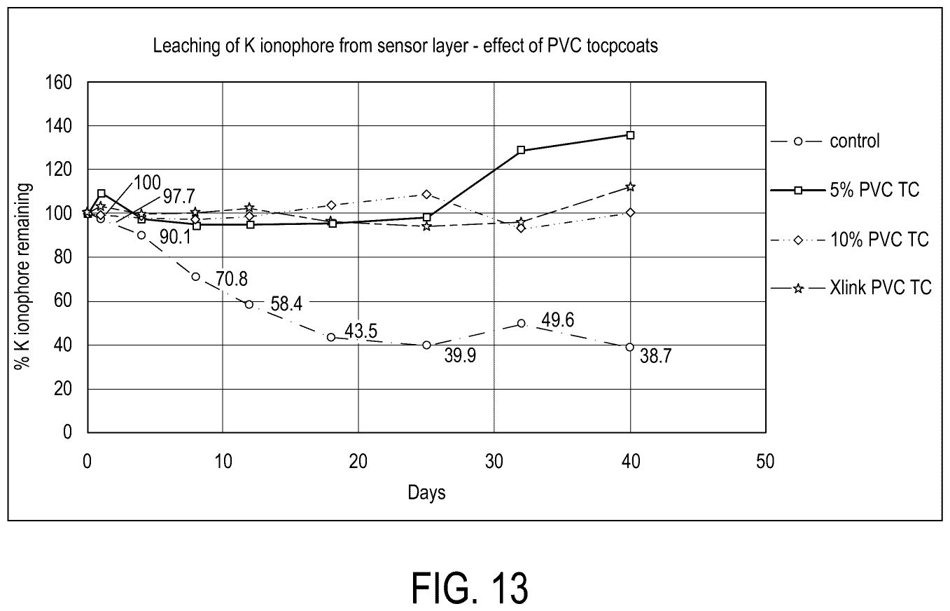

FIG. 13 is a graph showing leaching, or lack thereof, of ion-selective sensor molecules (e.g., potassium ionophore) from the indicator layer 1104 comparing different film-based sensor samples with or without a barrier film (e.g., barrier layer 1106). The control was a film-based sensor having potassium ionophore in the indicator layer 1104 without a barrier film. The samples labeled “5% PVC TC”, “10% PVC TC”, and “Xlink PVC TC” corresponded to film-based sensors having potassium ionophore in the indicator layer 1104, and additionally having a barrier layer 1106 adjacent the indicator layer 1104 formed by using a solution having 5% PVC, 10% PVC, and crosslinked PVC, respectively.

As shown in FIG. 13, each of the film-based sensor samples having a barrier layer 1106 show significant improvement in preventing leaching of the sensor molecules from the indicator layer 1104 compared to the control sample having no barrier layer. Each of the three samples having the barrier layer 1106 has no loss of sensor molecules over the course of 40 days. In other words, none of the three samples having the barrier layer 1106 has significantly less than 100% of sensor molecules remaining over the course of 40 days. The glass transition temperature of polyvinyl chloride (PVC) is typically between 70° C. and 90° C. Without wishing to be bound by theory, it appears that a barrier film including unplasticized PVC is effective at essentially stopping leaching of sensor molecules (e.g., ionophores) due in part to the fact that at body temperature (i.e., around 37° C.) the polymer is in its glassy state and has lower water permeability compared to a polymer below its glass transition temperature.

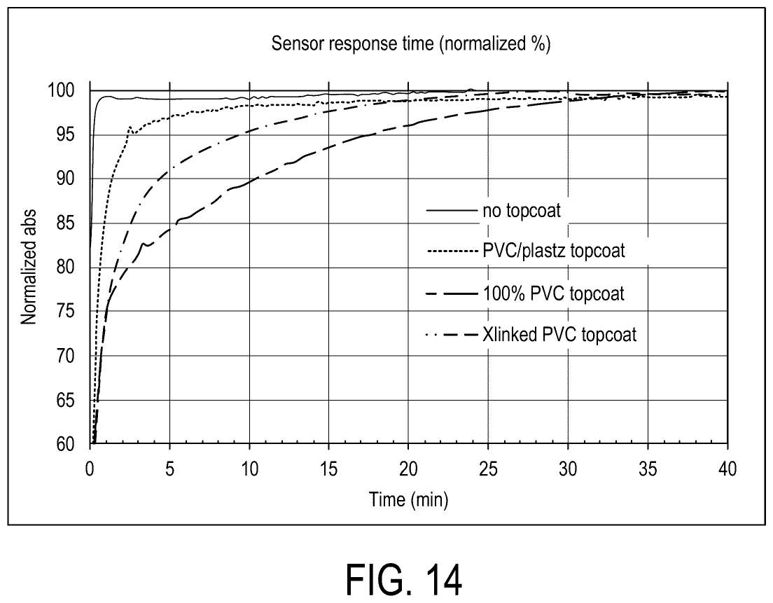

FIG. 14 is a graph showing sensor response time comparing various film-based sensor samples with or without a barrier film (e.g., barrier layer 1106). The y-axis is “normalized abs” showing percentage of light absorption as measured by a UV vis machine, and the x-axis is duration of time in minutes. As shown in FIG. 14, the control sample having no barrier layer has a response time of less than 1 minute to reach 95% absorption, sample having a mixture of PVC and plasticizer in the barrier layer 1106 has a response time of about 2-3 minutes to reach 95% absorption, sample having 100% PVC in the barrier layer 1106 has a response time of about 10 minutes to reach 95% absorption, and sample having crosslinked PVC in the barrier layer 1106 has a response time of about 17 to 18 minutes to reach 95% absorption. As such, all the samples having a barrier layer 1106 have good and/or acceptable response time. Therefore, as demonstrated by FIGS. 13 and 14, the barrier layer 1106 including PVC or crosslinked PVC do not significantly slow down sensor response time while effectively preventing leaching of sensor molecules from the indicator layer 1104.

### Methods

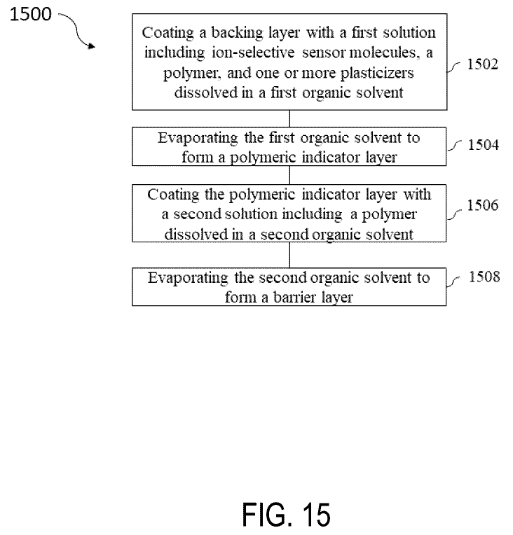

FIG. 15 is a flow diagram of an example method 1500 for making part of the chemical sensor described herein. One or more steps of method 1500 are optional and/or can be modified by one or more steps of other instances described herein. Additionally, one or more steps of other instances described herein may be added to the method 1500.

At step 1502, the method 1500 includes coating a backing layer with a first solution including ion-selective sensor molecules and the polymer dissolved in a first organic solvent. Suitable materials for the backing layer, ion-selective sensor molecules, and polymer are described above with regard to FIG. 11. The organic solvent may include any organic solvent known and used in the art for dissolving polymeric materials for coating with subsequent solvent evaporation, for example, tetrahydrofuran (THF) and/or toluene. In some instances, the first solution further includes one or more plasticizers. In some instances, the plasticizer included in the first solution may be dioctyl phthalate (DOP), di-n-butyl phthalate (DBPH), dibutyl sebacate (DBS), o-nitrophenyl octyl phthalate, dioctyl sebacate (DOS), tricresylphosphate (TCP), dibutyl phthalate (DBP), nitrophenyl octyl phthalate, di-n-butyl phthalate (DBPH), dibutyl phosphate, tri-n-butyl phosphate (TBP), bis(1-butylpentyl) adipate, bis(2-ethylhexyl) phthalate, tris(2-ethylhexyl) phosphate (TEHP), tricresyl phosphate (TCP), o-nitrophenyl octyl ether (NPOE), dinonyladipinate (DNA), bis(2-ethylhexyl) sebacate, bis(2-ethylhexyl) adipate (DOA), and bis(2-ethylhexyl sebacate) (BEHS), or any plasticizer known and used in the art for plasticizing polymers as disclosed herein.

At step 1504, the method 1500 includes evaporating the first organic solvent to form a polymeric indicator layer comprising the ion-selective sensor molecules, the polymer, and the one or more plasticizers.

At step 1506, the method 1500 includes coating on top of the polymeric indicator layer with a second solution comprising a polymer dissolved in a second organic solvent. In some instances, the polymer used in step 1506 is the same as the polymer used in step 1502. In some instances, the polymer used in step 1506 may include polyvinyl chloride (PVC), crosslinked PVC, polyimide, silicone rubber, polyurethane, acrylate, perfluoropolymers, or any polymer known and used in the art for ion-selective membrane materials. In some instances, the polymer used in both step 1502 and 1506 is PVC or crosslinked PVC.

The organic solvent used in step 1506 may be the same as or may be different from the organic solvent used in step 1502. The second organic solvent may include any organic solvent known and used in the art for dissolving polymeric materials for coating with subsequent solvent evaporation, for example, tetrahydrofuran (THF) and/or toluene. In some instances, the second solution does not include a plasticizer. In some instances, the second solution does not include ion-selective sensor molecules. In certain instances, the second solution includes the polymer and a plasticizer same as the ones used in the first solution, but does not include ion-selective sensor molecules.

The second solution may include from about 1 wt/wt % to about 20 wt/wt % of the polymer dissolved in the second organic solvent. In certain instances, the second solution includes from about 1 wt/wt % to about 2 wt/wt %, or from about 2 wt/wt % to about 3 wt/wt %, or from about 3 wt/wt % to about 4 wt/wt %, or from about 4 wt/wt % to about 5 wt/wt %, or from about 5 wt/wt % to about 6 wt/wt %, or from about 6 wt/wt % to about 7 wt/wt %, or from about 7 wt/wt % to about 8 wt/wt %, or from about 8 wt/wt % to about 9 wt/wt %, or from about 9 wt/wt % to about 10 wt/wt %, or from about 10 wt/wt % to about 11 wt/wt %, or from about 11 wt/wt % to about 12 wt/wt %, or from about 12 wt/wt % to about 13 wt/wt %, or from about 13 wt/wt % to about 14 wt/wt %, or from about 14 wt/wt % to about 15 wt/wt %, or from about 15 wt/wt % to about 16 wt/wt %, or from about 16 wt/wt % to about 17 wt/wt %, or from about 17 wt/wt % to about 18 wt/wt %, or from about 18 wt/wt % to about 19 wt/wt %, or from about 19 wt/wt % to about 20 wt/wt % of the polymer dissolved in the second organic solvent, or within any range encompassing any two of these values as endpoints.

At step 1508, the method 1500 includes evaporating the second organic solvent to form the migration-limiting barrier layer adjacent the polymeric indicator layer. In some instances, where the second solution includes a polymer dissolved in a solvent, and no additional plasticizers, the resulting migration-limiting barrier layer includes 100% of the polymer after solvent evaporation. In certain instances, where the second solution includes both a polymer and a plasticizer dissolved in a solvent, the resulting migration-limiting barrier layer includes a mixture of polymer and plasticizer after solvent evaporation. In some instances, the migration-limiting barrier layer has a thickness less than the thickness of the polymeric indicator layer. In some instances, the migration-limiting barrier layer has a thickness of less than 5 micrometers such as to not significantly increase sensor response time while preventing leaching/migration of the ion-selective sensor molecules from the indicator layer. The thickness of the barrier layer may be adjusted by adjusting the concentration of polymer in the second solution. Higher concentration of polymer in solution results in a thicker barrier layer after solvent evaporation.

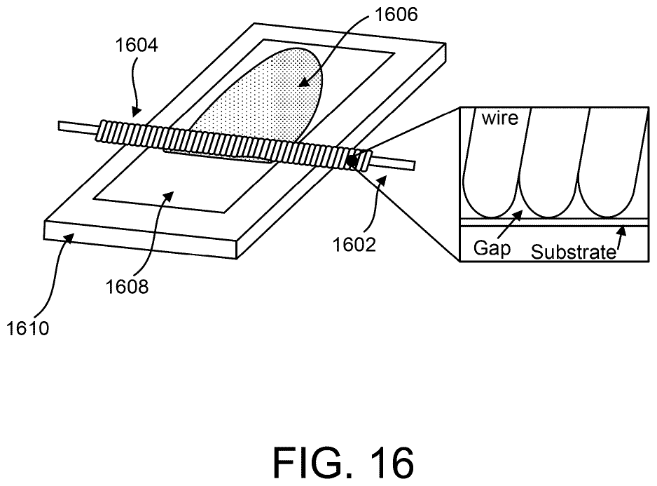

The coating steps (e.g., steps in method 1500 may be conducted via any means known to a skilled person in the art, for example but limited to, dip coating, spray coating, spin coating, and/or rod coating. FIG. 16 shows an exemplary setup for using rod coating to form a film-based sensor (e.g., film-based sensor 1100). As shown in FIG. 16, a coating rod 1602 having wire coils 1604 wrapped around the rod 1602 is used to spread out sensor coating solution 1606 (e.g., solution including ion-selective sensor molecules and/or polymer) on a base film 1608 (e.g., PET backing layer of a film-based sensor) on a glass plate 1610. One or more steps of the method 1500 may be conducted using the setup as depicted in FIG. 16. The thickness of the resulting film may be controlled by using different sized coating rods 1602 or wire coils 1604 as well as by varying concentration of the sensor coating solution 1606.

Various modifications and additions can be made to the exemplary instances discussed without departing from the scope of the present invention. For example, while the instances described above refer to particular features, the scope of this invention also includes instances having different combinations of features and instances that do not include all of the described features. Accordingly, the scope of the present invention is intended to embrace all such alternatives, modifications, and variations as fall within the scope of the claims, together with all equivalents thereof.

We claim:

1. A chemical indicator comprising:
a plurality of indicator beads having embedded therein ion-selective sensor molecules; and
a hydrogel surrounding the plurality of indicator beads, the hydrogel including a hydrophilic polymer bonded to a crosslinked network.

2. The chemical indicator of claim 1,
wherein the hydrophilic polymer comprises polyethylene glycol (PEG);
wherein the hydrogel is formed by crosslinking a functionalized PEG with a crosslinker to form the crosslinked network.

3. The chemical indicator of claim 2, wherein the functionalized PEG is selected from the group consisting of a mono-functional PEG, a bi-functional PEG, a multi-functional PEG, and a functionalized star PEG.

4. The chemical indicator of claim 2, wherein the crosslinker comprises an acrylamide selected from the group consisting of a mono-functional acrylamide monomer and a bi-functional acrylamide monomer.

5. The chemical indicator of claim 4, wherein the mono-functional acrylamide monomer is selected from the group consisting of N-Isopropylacrylamide, N,N-dimethylacrylamide, N-(3-(Dimethylamino)propyl)acrylamide, 6-Acrylamidohexanoic acid, N-(Tris(hydroxymethyl)methyl)acrylamide, N-(2-Aminoethyl)acrylamide hydrochloride, N-(2-Amino-2-oxoethyl)-2-propenamide, and Acryloylglycine.

6. The chemical indicator of claim 4, wherein the bi-functional acrylamide monomer is selected from the group consisting of 1,4-Bis(acryloyl(piperazine, Ethylenebisacrylamide, N,N′-(1,2-Dihydroxyethylene)bisacrylamide, N,N′-1,3-Propanediylbis(2-propenamide), N,N′-1,6-Hexanediylbis(2-propenamide), N,N′-1,4-Butanediylbis(2-propenamide), N,N′-(Oxybis(2,1-ethanediyloxy-3,1-propanediyl))bis(2-propenamide), and N,N′-(1,3-Phenylenebis(methylene))bis(2-propenamide).

7. The chemical indicator of claim 1, wherein the hydrogel is formed by crosslinking a first functionalized PEG with a second functionalized PEG to form the crosslinked network.

8. The chemical indicator of claim 7, wherein the first functionalized PEG is a mono-functional PEG acrylamide and the second functionalized PEG is a bi-functional PEG acrylamide.

9. The chemical indicator of claim 2, wherein the crosslinker is a multi-arm activated crosslinker.

10. The chemical indicator of claim 9, wherein the multi-arm activated crosslinker comprises an activated ester.

11. The chemical indicator of claim 9, wherein the multi-arm activated crosslinker comprises an amine.

12. The chemical indicator of claim 2, wherein the crosslinker comprises a diisocyanate.

13. The chemical indicator of claim 2, wherein the crosslinked network comprises a polyacrylate network, a polyacrylamide network, a polyamide network, or a polyurethane network.

14. A chemical indicator comprising:
a plurality of indicator beads having embedded therein ion-selective sensor molecules; and
a hydrogel surrounding the plurality of indicator beads, the hydrogel including a hydrophilic polymer interpenetrated with a crosslinked network.

15. The chemical indicator of claim 14, wherein the hydrogel is formed by polymerizing a functionalized PEG into the crosslinked network.

16. The chemical indicator of claim 15, wherein the functionalized PEG is a functionalized star PEG having more than two arms.

17. The chemical indicator of claim 16, wherein the functionalized PEG is physically entangled in the crosslinked network through a hydrogen bond and/or a non-covalent bond.

18. The chemical indicator of claim 17, wherein the crosslinked network comprises polyacrylates, polyacrylamide, polyamide, polyurethane, polyurea, or a combination thereof.

19. A chemical sensor cassette comprising:
an optical reflector; and
the chemical indicator of claim 1.

20. An apparatus comprising:
an optical feedthrough that includes:
a bottom portion,
a side wall portion surrounding a periphery of the bottom portion to create a well, and
a first window and a second window formed through the bottom portion of the optical feedthrough; and
a chemical sensor cassette that is at least partially positioned in the well and that includes the chemical sensor cassette of claim 19.
